(19) United States
(12) Patent Application Publication
(10) Pub. No.: US 2026/0144495 A1
(43) Pub. Date: May 28, 2026

(54) Machine Learning and Artificial Intelligence Enabled Medical Device System

(71) Applicant: Sureshbabu Sankararaj, Okemos, MI (US)

(72) Inventor: Sureshbabu Sankararaj, Okemos, MI (US)

(21) Appl. No.: 18/960,995

(22) Filed: Nov. 26, 2024

(51) Int. Cl. A61B 5/00; A61B 5/0205; G16H 10/60; G16H 20/90; G16H 40/67; G16H 50/20

(52) U.S. Cl. CPC A61B 5/7267; A61B 5/0205; A61B 5/681; G16H 10/60; G16H 20/90; G16H 40/67; G16H 50/20; A61B 2560/0242

(57) ABSTRACT

A machine learning and A.I. enabled medical device system 1 having a machine learning model analyzer integrated with the portable medicine device which controls various sensors in the device and collects data on real-time basis and select an appropriate machine learning model to facilitate and manage a holistic prescription instruction and machine learning and A.I. enabled medical device medication administration schedule. Model analyzer in turn receives plurality of approved Holistic prescription models, Medicine identifier models generated by the machine learning device utilizing sensors in the device and mobile app user input data refined by AI Assistant. The medicine container, which contains a medication, is removably coupled to a wearable mount wherein some embodiments, the wearable mount is a wristband configured to secure the medicine container to a wrist of a user.

### FIELD OF THE INVENTION

The present invention generally relates to a medical device. More specifically, the present invention is a machine learning and artificial intelligence enabled medical device having a portable medicine device including a plurality of sensors, a plurality of model generators and Model analyzers, a mobile application and an artificial intelligence (A.I.) assistant tool facilitating medication administration schedules.

### BACKGROUND OF THE INVENTION

The current landscape of medication administration contains limitations that hinder the effective management of medication schedules and adherence to holistic health prescriptions. Existing pill storage solutions, such as pillboxes and mobile medication reminder applications, are designed with static functionality and offer little flexibility for personalized care based on real-time health data. These systems operate on predetermined prescription schedules, set tolerance limits and dosage requirements, which are inadequate for users with complex health needs requiring adaptive, comprehensive healthcare.

Traditional medication management devices rely heavily on manual interactions, such as opening pill containers or relying on basic reminder alarms. There is little to no integration between the device carrying the medication and any external applications that provide medication reminders, leaving a disconnect between what the user is reminded to do and the actual administration of the medication

Traditional medication management devices heavily rely under the assumption that only medicine intake at set times or set tolerance values will improve the health conditions of the user and does not consider other human activities which affects the human body health parameters, such as type of dietary and nutritional values of the food intake, supplemental and combination effect of it with the medicine intake, combination of plurality of medication taken and its health effects, variable dosage medicine intake, timing of the medicine intake, type and amount of exercise done and its health effects, Stress and sleep patterns and its effect on the health and medicine efficiency, environmental factors and its effect on the individual health data, alternate treatments and many more or combinations thereof, which in turn acts a medicine itself. As a result, existing solutions fall short in providing holistic health care prescription and personalized medication management that will improve the medication efficiency, leading to inefficiencies and a higher likelihood of medication errors.

Existing devices have no useful art to continuously learn the user health effects and medicine efficiencies based on a holistic set of user's daily activities and combinations thereof as mentioned above and finding optimized model patterns which improves the user's health data by constantly learning user's health response patterns and recommending holistic prescriptions where in medicine intake instruction is just a part of the holistic prescriptions model. Device generated holistic prescriptions models includes optimized, personalized and sequenced real-time instructions on human activities, lifestyle changes and medication administration as per advanced machine learning models implemented in this device. In this invention the device will dynamically adjust Holistic prescription model, model parameters and instruction for the user and the device based on the real-time user health data and previously learnt user health response machine learning models for overall improvement of user's health metrics wherein the present invention overcome the limitations of setting up of predetermined Medicine intake timings and set tolerance limits of the existing device.

The present invention is designed to address these challenges by introducing a machine learning (ML) integrated wearable device and a mobile application system that manages and facilitates medication administration while providing a holistic prescription model to individual user's specific health needs. This system combines a wearable portable Medicine device with machine learning device, ML Model analyzer and a holistic prescription mobile application with AI assistant to create user-specific holistic prescription and medication schedules that adapt in real time based on user's health metrics and biometric inputs.

One of the major limitations of the prior art is their lack of versatility. Traditional pillboxes are rigid in design, restricting users to either use as a static box or a wearable watch with little flexibility in how they carry and administer their medication. The machine learning and A.I. enabled medical device offers a solution by allowing users to attach the portable medicine container to both static holders and mobile holders, providing flexibility in how they store and access their medication. This adaptable design ensures that medication is always available and synchronized with the holistic prescription mobile app for seamless medicine administration.

Another issue with current medication systems is the difficulty users with vision impairments or dexterity issues face when interacting with small interactive screens on wearable devices. The present invention overcomes this problem by allowing users to control the device through a phone, tablet, or laptop, giving them access to a larger screen and making it easier to interact with the holistic prescription mobile app and follow medication instructions.

Traditional medication administration is further hindered by the lack of coordination between apps and pill-carrying devices. Existing apps run independently, sending reminders to users without confirming whether the medication is available in the device or whether the user has taken the correct dosage. The machine learning and A.I.

enabled medical device resolves this issue by identifying the medicine availability and prescription instructions syncing with both the holistic prescription mobile app and the wearable machine learning and A.I. enabled medical device, ensuring that users have the correct medication information and the guidance they need to take it correctly.

Battery dependency is another challenge faced by the prior art, as many rely on constant charging. If either the device or the app runs out of charge, users are left without crucial information about their medication regimen. The present invention addresses this by offering redundancy; if the device runs out of battery, users can still access instructions through the app, and vice versa. This ensures continuous access to medication instructions without interruptions.

A significant shortcoming in current designs is the lack of integration with users'health records or real-time inputs based on current user biometric readings and inputs from doctors. Most medication management systems do not provide a way for doctors to monitor their patients'adherence to prescribed regimens and adjust treatment based on real-time biometric data of the user. The machine learning and A.I. enabled medical device overcomes this limitation by integrating lab results, doctor-approved prescription models, and monitoring real-time user health biometric data into the holistic prescription mobile app. This allows doctors and caregivers to monitor the user's compliance with the prescribed regimen and adjust the treatment model in real time. The system also includes a built-in feature for registered caregivers and doctors to contact the user through the device if non-compliance is detected, further ensuring patient safety and adherence.

Another critical issue with current pill storage systems is the lack of precision in monitoring medication intake. Traditional devices use basic methods like detecting medicine intake when a container is opened or estimating weight changes to determine whether a user has taken their medication. These methods are error-prone and assumption-based, which can lead to inaccuracies in medication administration. The machine learning and A.I. enabled medical device employs advanced machine learning models and AI based medicine identifier techniques to accurately identify the medication available inside the portable medicine container. This ensures that the correct dosage and medication are identified before and after medicine administration, reducing the likelihood of errors.

Moreover, existing medication apps follow rigid, static schedules, offering little personalization based on an individual user's health needs. These systems do not consider varying human activities factors and other variables which affect the user's normal biometric parameters, and often deliver reminders based solely on preset times and dosages. The machine learning and A.I. enabled medical device provides personalized reminders that are adjusted based on a holistic health prescription model considering varying factors such as medication intake, medication combination, variable timings and medicine dosages, nutrition & supplemental intakes, exercise, changes in environment variables and many more variables or combinations thereof which affects users health parameters values.

Existing pillboxes doesn't co-ordinate full lifecycle of medicine administration.

The machine learning and A.I. enabled medical device provides color-coded reminders and display real-time alerts for users on when to take their medication, when to pause taking medication, when to refill the storage container, and what dosage to take based on real-time health data and doctor-approved holistic prescription models and instructions to improve or normalize the user biometric readings.

In addition to providing better health outcomes, the present invention addresses privacy concerns that plague current digital health solutions. In many existing systems, user data is stored in large, cloud-based databases, raising concerns about security breaches. The machine learning and A.I. enabled medical device, however, stores user data locally on the machine learning and A.I. enabled medical device and other user registered device or personal devices like their phone, tablet, or laptop. This localized health data storage ensures that the user retains control over their personal data, which is shared with doctors or caregivers only with the user's explicit consent, in compliance with HIPAA regulations. Only anonymous data will be stored in the cloud to generate useful holistic prescription models.

Another shortcoming of the prior art is their limited customization options. Most pillboxes and medication apps offer only basic reminder functions with predetermined timings and tolerance limits and do not provide the flexibility needed to create personalized, holistic prescriptions for users. The machine learning and A.I. enabled medical device allows full customization, enabling users to configure settings on any input and output parameters collected or transmitted by the device which improves the user's health data along with care provider feedback inputs in order improve the accuracy of the holistic prescription for the users. This creates a holistic health management system tailored to the individual specific healthcare needs.

Finally, the lack of machine learning models in the prior art hinders the understanding of user's health care needs and user health response patterns on real time and personalized basis where in the user health response constantly change over a period and there by needing to adjust dynamically the holistic prescription plans based on user's healthcare needs. Most existing systems do not incorporate machine learning or AI to suggest holistic prescription models. The machine learning and A.I. enabled medical device fills this gap by incorporating advanced ML algorithms that continuously learn on the user's health response data and generate user specific holistic prescription models and holistic prescription instructions based on real-time and previously recorded user response model data. Doctors can review these models, approve them, and adjust treatment regimen, accordingly, providing a more dynamic and personalized approach to individual user's health care needs.

In conclusion, the present invention addresses the significant limitations of current medication administration systems by offering a versatile, personalized, and data-driven approach. By integrating machine learning device, a model analyzer, mobile application, A.I. assistant, real-time health monitoring, and doctor-approved prescription models to the machine learning and A.I. enabled medical device system 1 provides users with a holistic prescription model tailored to user's individual health care needs. This invention ensures improved medication adherence, better health outcomes, and enhanced user convenience while maintaining the highest standards of data privacy and security.

### BRIEF DESCRIPTION OF THE DRAWINGS

FIG. 1 is a table of the components machine learning and artificial intelligence enabled medical device system, referred to herein as the present invention.

FIG. 2 is a profile view of the portable medicine device of the present invention.

FIG. 3 is a perspective view of the portable medicine device of the present invention.

FIG. 4 is a table of the components of the medicine container of the present invention.

FIG. 5 is an exploded perspective view of the portable medicine device of the present invention.

FIG. 6 is a top view of the internal cavity of the medicine container of the present invention.

FIG. 7 is a table of the components of the interior cavity of the present invention.

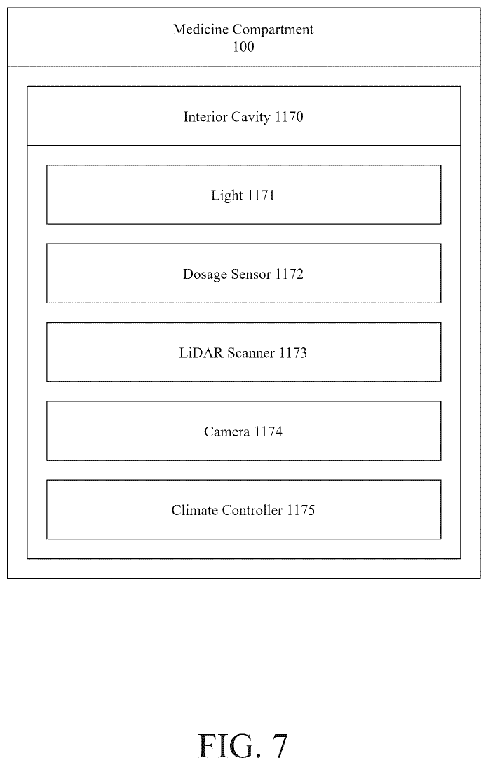

FIG. 8 is a table of the computer executable methods of the present invention.

FIG. 9 is a table of the components of the indicator interface of the portable medicine device of the present invention.

FIG. 10 is a table of the components of the indicator interface of the portable medicine device of the present invention, wherein the medicine container comprises the indicator interface.

FIG. 11 is a table of the components of the indicator interface of the portable medicine device of the present invention, wherein the wearable mount comprises the indicator interface.

FIG. 12 is a table of the communication device of the present invention.

FIG. 13 is a table of the plurality of sensors of the present invention.

FIG. 14 is a perspective view of one embodiment of the present invention.

FIG. 15 is a perspective view of one embodiment of the docking station of the present invention.

FIG. 16 is a perspective view of an alternate embodiment of the docking station of the present invention.

FIG. 17 is a perspective view of an additional alternate embodiment of the docking station of the present invention.

FIG. 18 is a table of the components of the docking station of the present invention.

FIG. 19 is a network diagram of the present invention.

FIG. 20 is a table of the mobile device of the present invention.

FIG. 21 is a table of the plurality of modules of the present invention.

FIG. 22 is a table of the analyzer module comprising an A.I. assistant.

FIG. 23 is a process diagram of the holistic prescription machine learning model generator method of the present invention.

FIG. 24 is a process diagram of the medicine identifier machine learning model generator method of the present invention.

FIG. 25 is a process diagram of the holistic prescription selection and medicine identifier process method of the present invention.

### DETAIL DESCRIPTIONS OF THE INVENTION

All illustrations of the drawings are for the purpose of describing selected versions of the present invention and are not intended to limit the scope of the present invention.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Additionally, it is important to note that each term used herein refers to that which an ordinary artisan would understand such term to mean based on the contextual use of such term herein. To the extent that the meaning of a term used herein-as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

Other technical advantages may become readily apparent to one of ordinary skill in the art after review of the following Figures and description. It should be understood at the outset that, although exemplary embodiments are illustrated in the Figures and described below, the principles of the present disclosure may be implemented using any number of techniques, whether currently known or not. The present disclosure should in no way be limited to the exemplary implementations and techniques illustrated in the drawings and described below.

Unless otherwise indicated, the drawings are intended to be read together with the specification and are to be considered a portion of the entire written description of this invention. As used in the following description, the terms “horizontal”, “vertical”, “left”, “right”, “up”, “down” and the like, as well as adjectival and adverbial derivatives thereof (e.g., “horizontally”, “rightwardly”, “upwardly”, “radially”, etc.), simply refer to the orientation of the illustrated structure as the particular drawing Figure faces the reader. Similarly, the terms “inwardly,” “outwardly” and “radially” generally refer to the orientation of a surface relative to its axis of elongation, or axis of rotation, as appropriate.

The present disclosure includes many aspects and features. Moreover, while many aspects and features relate to, and are described in the context of a machine learning and A.I. enabled medical device system 1, embodiments of the present disclosure are not limited to use only in this context.

In the context of the present invention, a holistic prescription refers to a set of prescriptions and controls which affects human biometric parameters in combination with the medication taken and its effects on the overall health parameter improvements. Holistic prescription includes the instruction on the type of dietary and nutritional values of the food intake, the instruction on the supplemental medications and its intake methods, the instruction on the combination of medication intake, the instruction on the variable medicine dosages and medicine intake details, the instruction on the variable timings of the medicine intake, the instruction on the type and the amount of exercise to be done, the instruction on the stress reduction and sleep pattern improvements, the instruction on adjusting environmental factors, the instruction on the alternate treatment plans and many more or combinations thereof as per the sequence and timings of the instructions.

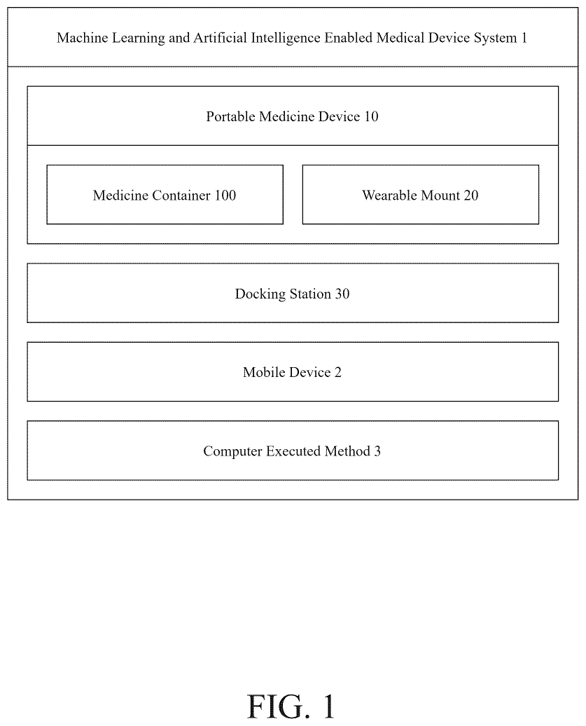

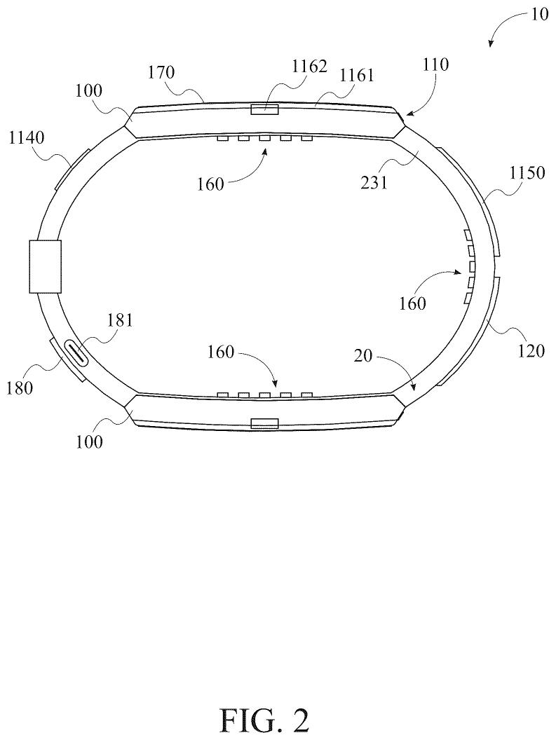

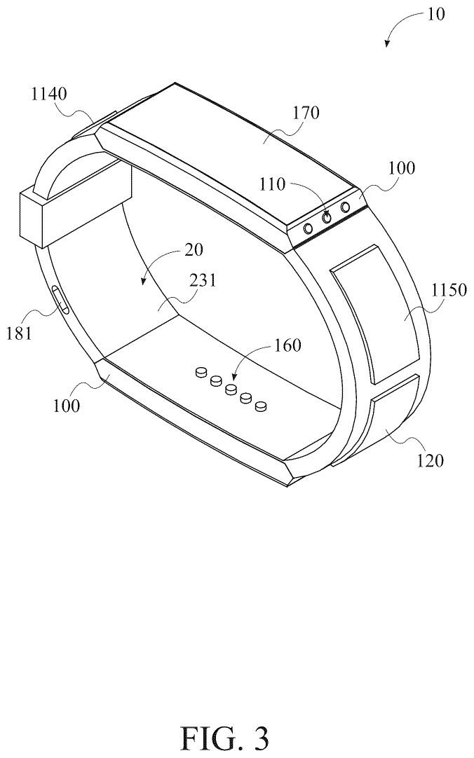

As shown in FIG. 1, the present invention is a machine learning and A.I. enabled medical device system 1 comprising a portable medicine device 10, a mobile device 2, and a computer executed method. In the preferred embodiment of the present invention, as shown in FIG. 1, FIG. 2, and FIG. 3, the portable medicine device 10 is composed of a medicine container 100 and a wearable mount 20. In the preferred embodiment of the present invention, machine learning and A.I. enabled medical device system 1, further comprises a docking station 30 wherein the medicine container 100 in the portable medicine device 10 removably couples to said docking station 30. Moreover, within the preferred embodiment of the present invention, machine learning and A.I. enabled medical device system 1, the portable medicine device 10, the mobile device 2, and the docking station 30 execute the computer executable method 3.

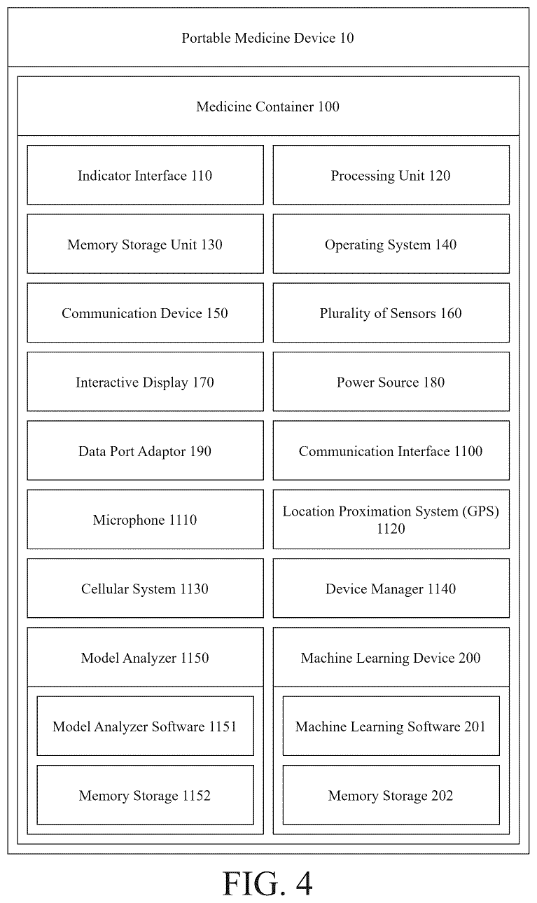

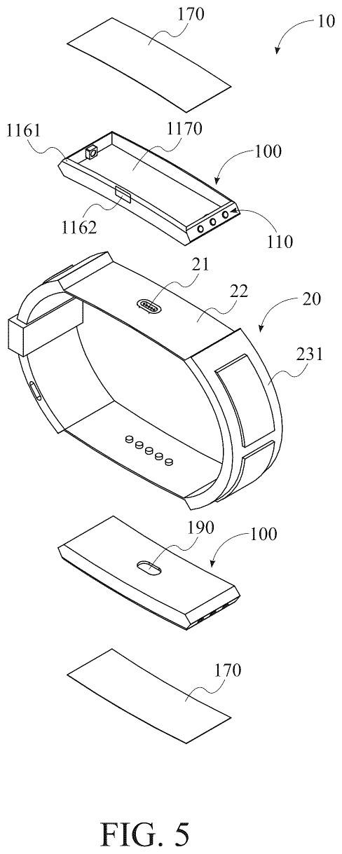

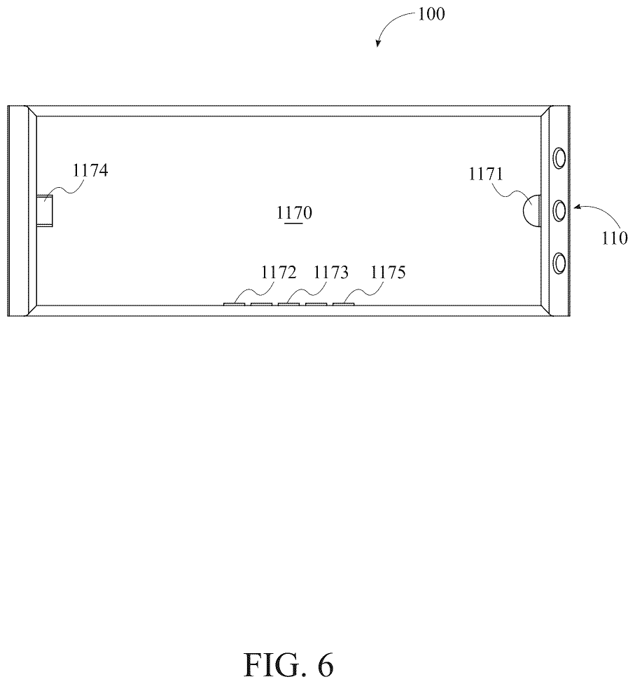

In the preferred embodiment of the present invention, the machine learning and A.I. enabled medical device system 1, as shown in FIG. 4 and FIG. 5, comprise the portable medicine device 10 wherein said portable medicine device 10 comprises the medicine container 100, also referred to herein as a medicine container 100. In the preferred embodiment of the present invention, as shown in FIGS. 4, 5, and 6 the medicine container 100 comprises an indicator interface 110, a processing unit 120, a memory storage unit 130, an operating system 140, a communication device 150, a plurality of sensors 160, an interactive display 170, a power source 180, a data port adaptor 190, a communication interface 1100, a microphone 1110, a location proximation system 1120 (GPS), a cellular system 1130, a device manager 1140, a machine learning device 200 and a model analyzer 1150. Furthermore, within the context of the present invention, the power source comprises a charging port 181. In the context of the present invention, components of the medicine container 100 can be distributively assembled between the medicine container 100 and wearable mount 20 as shown in the FIGS. 2, 3, 4, 5 and 6 wherein some of the said components of the medicine container is integrated in the wearable mount and facilitates the control processes performed by the portable medicine device 10 as an integrated system. Additionally, within the preferred embodiment of the present invention, as shown in FIG. 6, the medicine container 100 further comprises an interior cavity 1170, wherein said interior cavity 1170 comprises a light 1171, a dosage sensor 1172, a lidar scanner 1173, a camera 1174, and a climate controller 1175. In the context of the present invention, the interior cavity 1170, in its intended use, contains a user's medication. Additionally, the interactive display receives an input from a user wherein said input manipulates a configuration and output by the model analyzer 1150 in the portable medicine device 10. Furthermore, in the preferred embodiment of the present invention, the indicator interface 110 and the interactive display 170 generates an output indicative of information pertaining to the interior cavity 1170 and a respective medication contained within said interior cavity 1170. In the context of the present invention, the processing unit 120 receives computer readable data, facilitates user and system inputs and system outputs, and executes system functions and the computer executable method 3. In the context of the present invention, the processing unit 120 executes instructions of a computer program, software, and integrated mobile application 40, such as arithmetic, logic, controlling, interfacing and input/output (I/O) operations. Additionally, within the context of the present invention, the location proximation system 1120 is a system configured to obtain and transmit the geographic location of the portable medicine device 10. In some embodiments of the present invention, the location proximation system 1120 is selected between a global system for mobile communications (GSM) location and a global positioning system (GPS) location. In the embodiments of the present invention, memory storage unit 130 is a storage device which stores all the loaded, collected, processed and received data. In the embodiments of the present invention, device manager 1140 controls and facilitates all the device control functionality of the device configured with the processing unit 120. Furthermore, within the context of the present invention, the data port adaptor 190 is a coupling connection allowing the medicine container 100 in the portable medicine device 10 to connect and access or be accessed by external devices, including the docking station 30 and the wearable mount 20, through a data port adaptor 190 and port connection 21, respectively. Additionally, within the context of the present invention, the plurality of sensors 160 are devices that reads various medicine identifier and biometric data, thereby communicating with a machine learning device 200 through the processing unit 120, the model analyzer 1150 through the processing unit 120, the mobile application 40 and the device manager 1140. Moreover, in the context of the present invention, the machine learning device 200 is a device which comprise of a machine learning software and a memory storage that executes, trains, and utilizes holistic prescription machine learning model generator a computer executable method 3 processed through the processing unit 120 which executes with collected user biometric sensor 161 data along with user inputs from plurality of modules in the mobile application 40 to generate holistic prescription machine learning models. Moreover, in the context of the present invention, additionally the machine learning device 200 is a device which comprise of a machine learning software and a memory storage that executes, trains, and utilizes medicine identifier machine learning model generator a computer executable method 3 processed through the processing unit 120 which utilizes medicine identifier sensor data to generate plurality of medicine identifier machine learning models. Moreover, in the context of the present invention, the model analyzer 1150 is a device which comprise of a model analyzer 1150 software and a memory storage that executes, analyze and utilizes a computer executable method 3 processed through the processing unit 120 to choose the best possible medicine identifier and holistic prescription model from the plurality of deployed medicine identifier and holistic prescription machine learning models stored in the portable device and based on real-time sensor data collected from the device user and pass the control parameters of the chosen model to the device manager 1140. Furthermore, in the context of the present invention, the battery is a power storage device wherein said power storage device maintains an electrical charge and releases said electrical charge, thereby providing power to the machine learning and A.I. enabled medical device system 1. In the context of the present invention, the device manager 1140 is a system wherein said system facilitates the control processes performed by the portable medicine device 10 and interfacing functions with the connected devices.

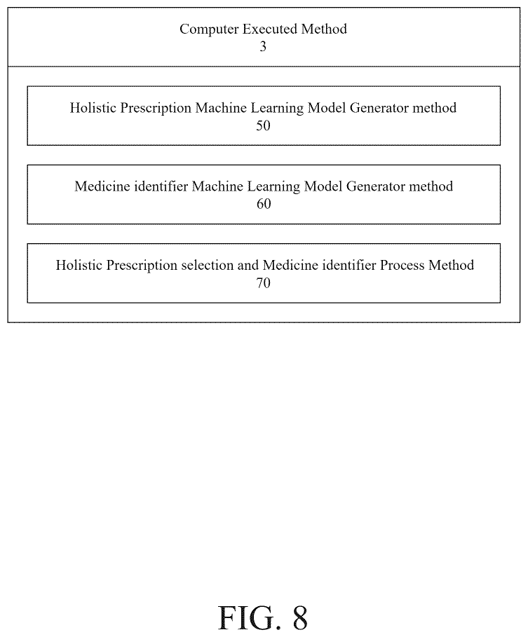

As shown in FIG. 2 and FIG. 5, in the preferred embodiment of the present invention, portable medicine device 10 comprises of the medicine container 100 comprises a door 1161 with interactive display 170 and a locking mechanism 1162, wherein the door 1161 provides accessibility to the interior cavity 1170 and the locking mechanism 1162 variably controlled by the device manager 1140 to allow access to said interior cavity 1170. As previously mentioned, in the preferred embodiment of the present invention, the interior cavity 1170, as shown in FIG. 8, also referred to herein as the interior cavity 1170, comprises the light 1171, the dosage sensor 1172, the lidar scanner 1173, the camera 1174, and the climate controller 1175. In the context of the present invention, the light 1171 illuminates the interior cavity 1170 of the medicine container 100. Furthermore, the climate controller 1175, as disclosed herein, regulates a temperature and a humidity of the interior cavity 1170 of the medicine container 100. In the context of the present invention, the camera 1174 along with the machine learning device 200 and model analyzer 1150 is configured to collect medicine identifier data from within the interior cavity 1170 of the medicine container 100. Moreover, in the context of the present invention, the dosage sensor 1172 is a device configured with the machine learning device 200 and model analyzer 1150 to obtain data from within the interior cavity 1170 of the medicine container 100 pertaining to the medication wherein said data obtained by the dosage sensor 1172 is a medication identification, a dosage metric of a medication, and data of the like pertaining to a medication. Likewise, in the preferred embodiment of the present invention, the lidar scanner 1173 is configured with camera 1174 and machine learning device 200 and model analyzer 1150 to detect medication within the interior cavity 1170.

In the preferred embodiment of the present invention, the portable medicine device 10 comprises the processor in the processing unit 120 whereby said processor executes a machine learning software in the machine learning device 200 and facilitated by the operating system 140 and memory storage unit 130,202, wherein said machine learning software includes a holistic prescription machine learning model generator method 50 as shown in FIG. 8 and medicine identifier machine learning model generator method 60 as shown in FIG. 8. In the preferred embodiment of the present invention, portable medicine device 10 further comprises of a model analyzer 1150 which executes a model analyzer 1150 software facilitated by the operating system 140 and memory storage unit 130,1152, wherein said model analyzer 1150 includes holistic prescription selection and medicine identifier process method 70 as shown in FIG. 8.

In the preferred embodiment of the present invention, the portable medicine device 10 comprises the model analyzer 1150 which executes the holistic prescription selection and medicine identifier process method and controls the plurality of sensors 160 to collect real-time sensor data to accurately identify the medicine inside the medicine container 100 from the plurality of trained and approved medicine identifier models stored in the portable medicine device 10 storage unit.

In the preferred embodiment of the present invention, the portable medicine device 10 comprises of a model analyzer 1150 which executes holistic prescription selection and medicine identifier process method and control the plurality of sensors 160 to collect the real-time biometric data and mobile application 40 user input data to dynamically determine the most appropriate holistic prescription model from the plurality of deployed holistic prescription machine learning models stored in the portable medicine device 10 storage unit. Furthermore, model analyzer 1150 records user response pattern which is transmitted to holistic prescription machine learning model generator in the machine learning device 200 for further learning and training of holistic prescription models for the user.

In the preferred embodiment of the present invention, the model analyzer 1150 will send the model parameters of the chosen holistic prescription model parameters to the device manager 1140 to control the chosen portable medicine device 10 interactive interface display screen and indicator interface 110 for the user to follow holistic prescription based on the selected holistic prescription model.

In the preferred embodiment of the present invention, the portable medicine device 10 provides holistic prescription instructions through the device interactive display 170 and indicator interface 110 to communicate to the user to follow holistic prescription instructions comprising of the instruction on the type of dietary and nutritional values of the food intake, the instruction on the supplemental medications and its intake methods, the instruction on the combination of medication intake, the instruction on the variable dosage and medicine intake details, the instruction on the timing of the medicine intake, the instruction on the type and the amount of exercise to be done, the instruction on the stress reduction and sleep pattern improvements, the instruction on adjusting environmental factors, the instruction on the alternate treatment plans and many more or combinations thereof and its sequence and timings of the instructions dynamically on real-time basis based on user's health metrics data.

In the context of the present invention, the interactive display 170 is a device configured to receive a user input and output display. In some embodiments of the present invention, the device interactive display 170 is a screen, wherein a user may navigate a graphical user interface. In alternate embodiments of the present invention, the interactive display 170 is an analog device, including a button, wherein said analog device receives and communicates a manual user input. In some embodiments of the present invention, the interactive display provides holistic medical instructions instructing a user of the holistic prescription medication administration on the display screen. Further, in the context of the present invention, the device interactive display 170 generates an output indicative of an actionable task intended to be performed by the user in accordance with the holistic prescription. In some embodiments of the present invention, interactive display 170 will be used for interactions between the user with the portable medicine device 10, the docking station 30, the mobile application 40 and the computer executable method 3.

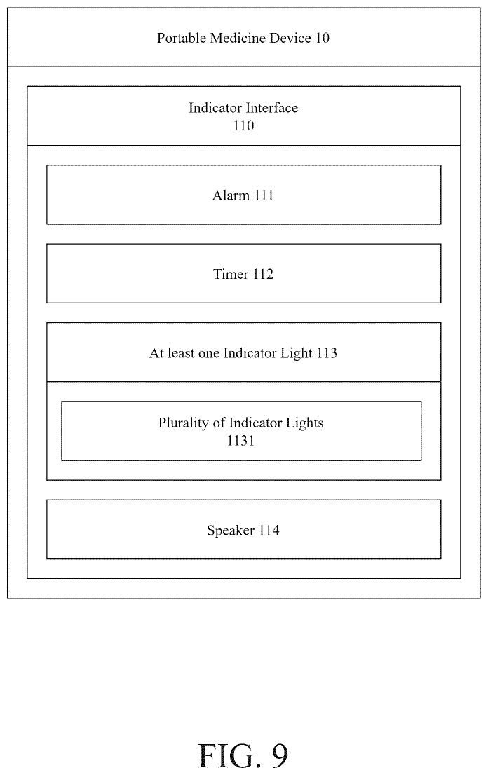

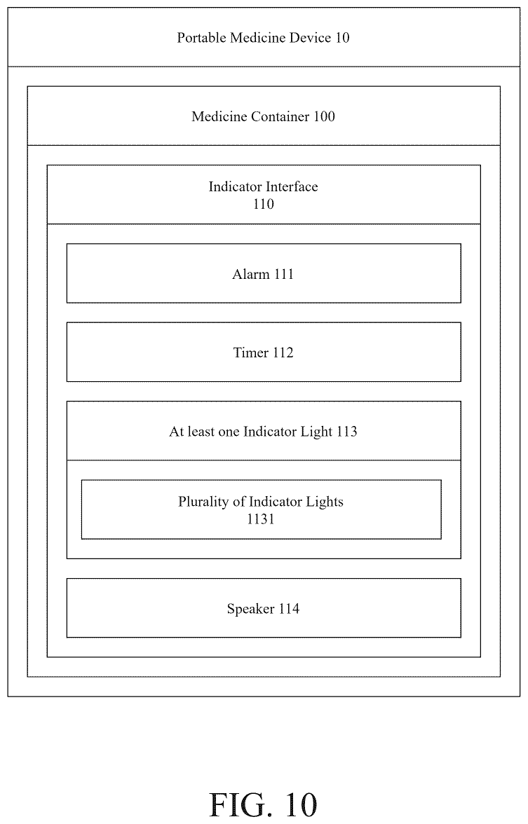

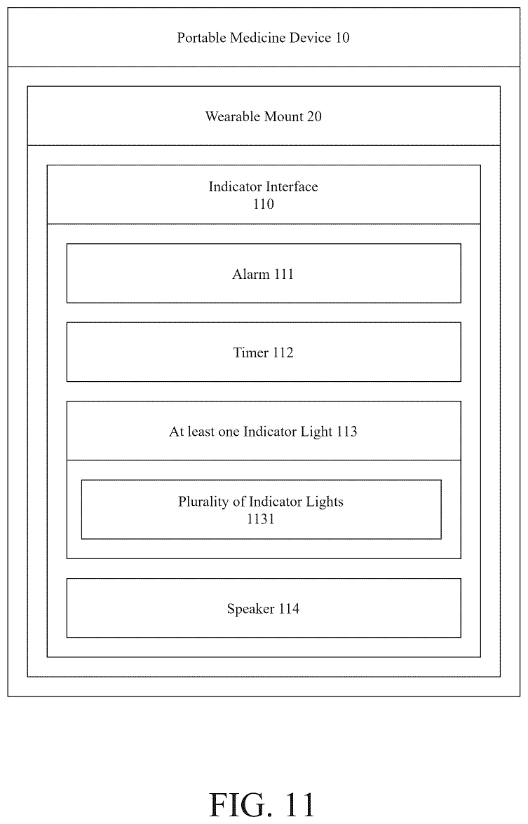

Furthermore, in the preferred embodiment of the present invention, the indicator interface 110 communicates to a user, according to a holistic prescription schedule, information pertaining to a medication contained within the medicine container 100. In some embodiments, the indicator interface 110 communicates to a user when to take a specific medication. In the preferred embodiment of the present invention, the indicator interface 110, as shown in FIGS. 9, 10, and 11 comprises an alarm 111, a timer 112, an at least one indicator light 113, and a speaker 114. In the preferred embodiment, the timer 112 controls a series of outputs generated by the alarm 111, the at least one light 113, and the speaker 114. Further, in the context of the present invention, the alarm 111 and the speaker 114 generate an output indicative of an actionable task intended to be performed by the user in accordance with the holistic prescription schedule. Further, in the preferred embodiment, at least one indicator light 113 generates an output indicative of a status wherein said status is representative of an availability of medicine contained within the interior cavity 1170 of the medicine container 100 per holistic prescription schedule.

Within the preferred embodiment of the present invention, the at least one indicator light 113 comprises a plurality of indicator lights 1131 wherein said plurality of indicator lights 1131 comprises three led lights. In the aforementioned embodiment, the three led lights comprise three unique colors (e.g. Red, yellow, and green), whereby each color is indicative of a unique status and desired action item intended to be taken by the user.

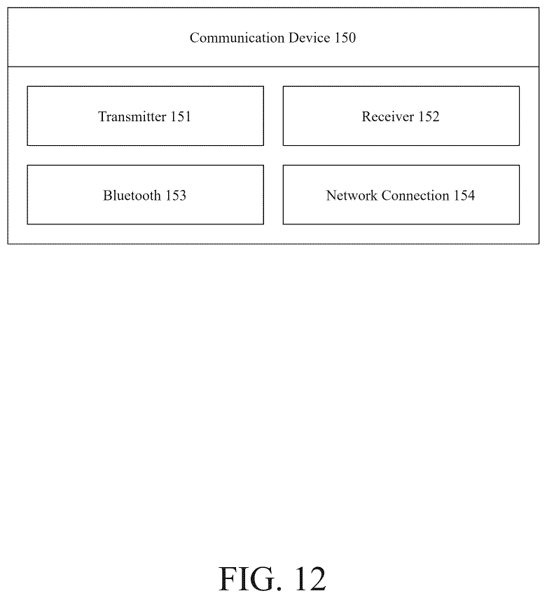

As shown in FIG. 12, in the preferred embodiment of the present invention, the portable medicine device 10, comprises the communication device 150 wherein said communication device 150 comprises at least one of a transmitter 151, a receiver 152, a Bluetooth module 153, and a network connection 154.

In the preferred embodiments of the present invention, the processing unit 120 is available in each of the portable medicine container 100 for it to independently process holistic prescription selection, medicine identifier and medicine administration functionality. In the preferred embodiments of the present invention, the processing unit 120 is available in the machine learning and A.I. enabled medical device system 1 for machine learning software to process machine learning model generators and to control the device functions. In the preferred embodiments of the present invention, as shown in FIG. 4, the processing unit 120, storage device 130, operating system 140 is available for the machine learning device 200 which comprise of machine learning software, a memory storage 202 to process machine learning model generators and to control the device functions through device manager 1140. In the preferred embodiments of the present invention, the processing unit 120, storage device 130, operating system 140 is available for the model analyzer 1150 which comprise of a model analyzer 1150 software, a memory storage 1152 to analyze machine learning models and to control the device functions through device manager 1140. In some embodiments of the machine learning and A.I. enabled medical device system 1, at least one processing unit 120 is available to process and control all the components in an integrated fashion of the machine learning and A.I. enabled medical device system 1.

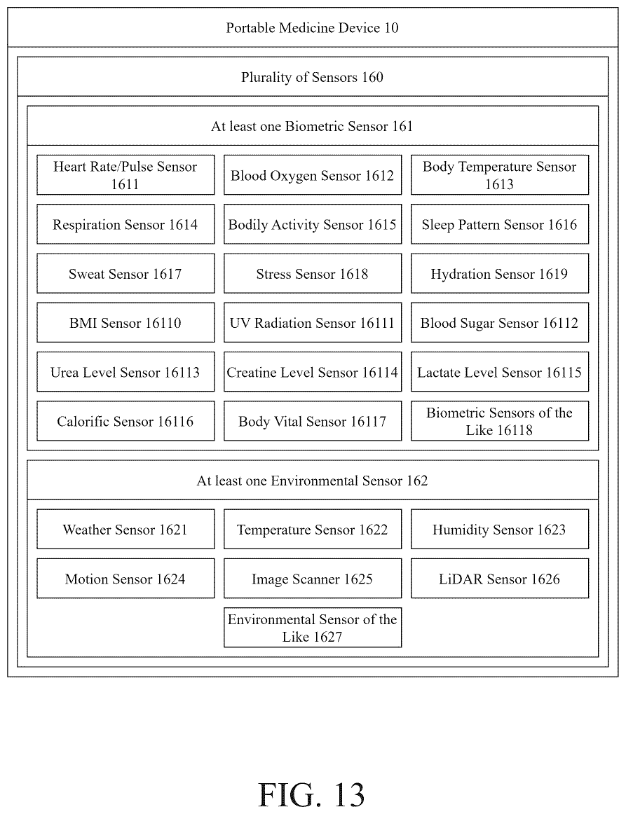

As shown in FIG. 13, in the preferred embodiment of the present invention, the plurality of sensors 160 comprises an at least one biometric sensor 161 and an at least one environmental sensor 162. In the preferred embodiment of the present invention, the at least one biometric sensor 161 is composed of at least one of: a heart rate/pulse sensor 1611, a blood oxygen sensor 1612, a body temperature sensor 1613, a respiration sensor 1614, a bodily activity sensor 1615, a sleep pattern sensor 1616, a sweat sensor 1617, a stress sensor 1618, a hydration sensor 1619, a BMI sensor 16110, a UV radiation sensor 16111, a blood sugar sensor 16112, a urea level sensor 16113, a creatine level sensor 16114, a lactate level sensor 16115, a calorific sensor 16116, a body vitals sensor 16117, and biometric sensors of the like 16118. Likewise, in the preferred embodiment of the present invention, the at least one environmental sensor 162 comprises at least one of: a weather sensor 1621, a temperature sensor 1622, a humidity sensor 1623, a motion sensor 1624, an image scanner 1625, a lidar sensor 1626, and an environmental sensor of the like 1627. In the preferred embodiment of the present invention, a date and time data is associated with each of the collected data values. Additionally, in the preferred embodiment of the present invention, plurality of sensors 160 collects data and communicates said data in a machine-readable format to the machine learning device 200, the model analyzer 1150, the mobile application 40 and the processing system.

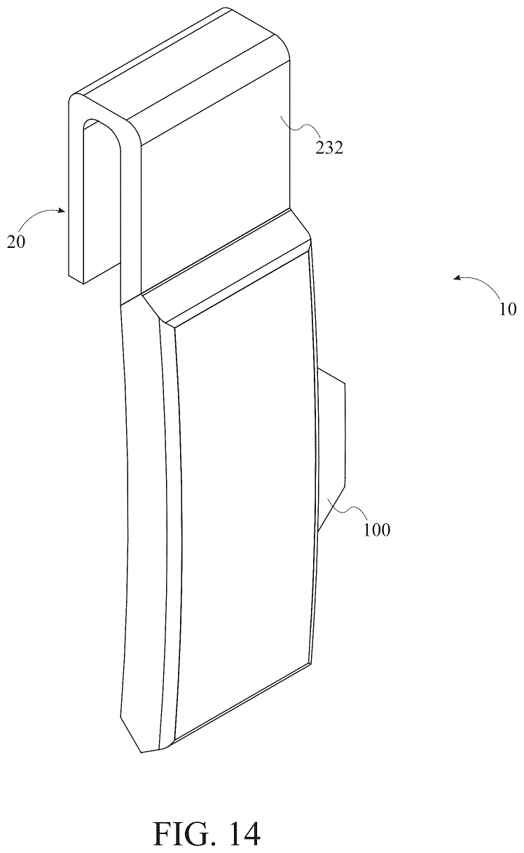

As shown in FIG. 14 in some embodiments of the present invention, the machine learning and A.I. enabled medical device system 1 further comprises a wearable mount 20 wherein the wearable mount 20 couples the portable medicine container 100 to a portion of the user's body. In the preferred embodiment of the present invention, the wearable mount 20 is a wristband 231 (shown in FIGS. 2 and 3) whereby the portable medicine device 10 is thereby adjustably secured to the wrist of a user. In some embodiments of the present invention, as shown in FIG. 2 and FIG. 3, a plurality of portable medicine containers 100 may be coupled to the wearable mount 20. In alternate embodiments of the present invention, as shown in FIG. 14, the wearable device is a clip 232 wherein the clip 232 comprises a j-shape configuration. In the preferred embodiment of the present invention, the portable medicine container 100 removably couples to the wearable mount 20.

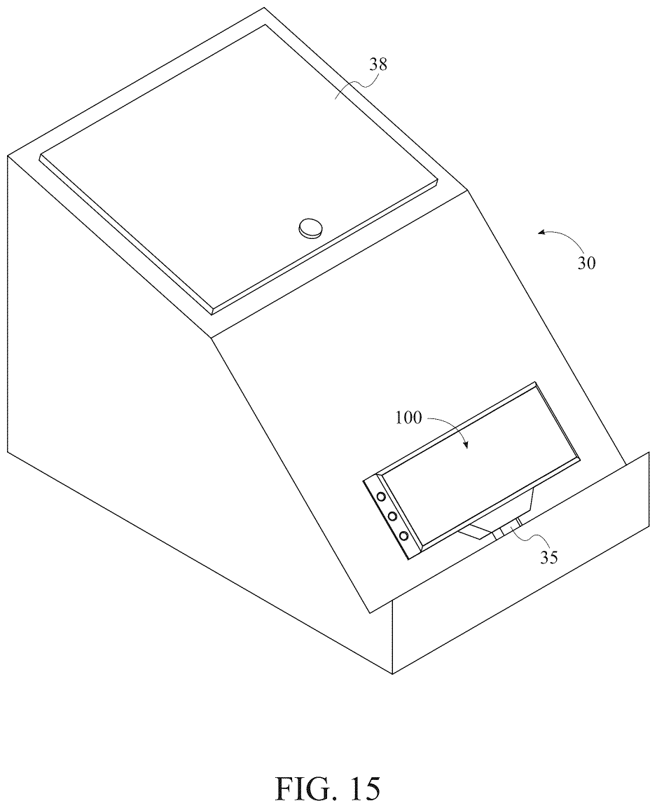

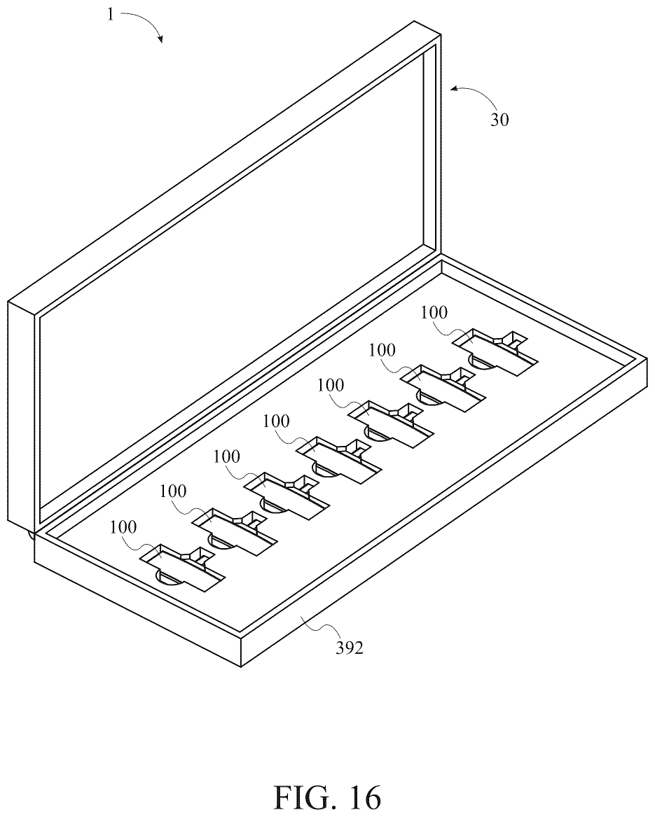

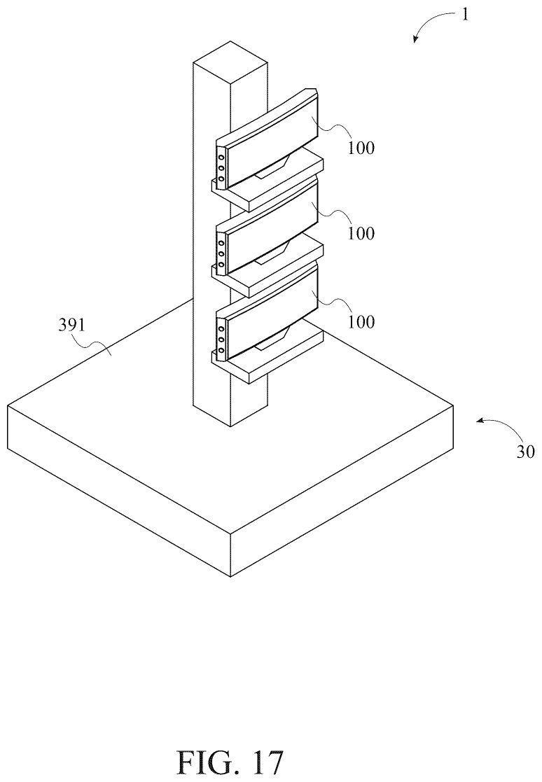

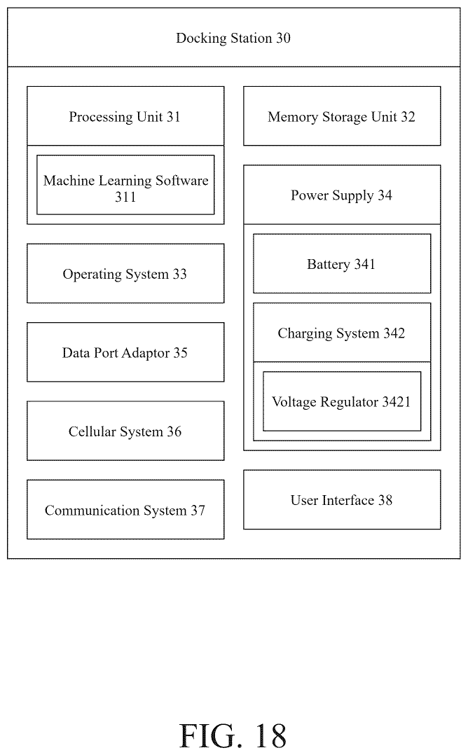

As shown in FIGS. 15, 16, and 17, in the preferred embodiment of the present invention, the docking station 30 is a device that couples to at least one portable medicine container 100. In the preferred embodiment of the present invention, the docking station 30, as shown in FIG. 15, comprises a user interface 38 and a data port adaptor 35. In the context of the present invention, the interface sends and receives various user configured input data. Furthermore, in some embodiments of the present invention, the docking station 30 communicates with a network 4 wherein the machine learning model is executed using data gathered by the portable medicine device 10, upon coupling the portable medicine container 100 to the docking station 30. In alternate embodiments, as shown in FIGS. 16 and 17, the docking station 30 may be configured in a case 392 and a stand 391 respectively. In some embodiments, the docking station 30 may be configured to receive a plurality of portable medicine containers 100 as a distinct portable medicine container 100 may be used for distinct purposes and occasions. For example, a user may possess one portable medicine container 100 for each day of the week or each section of a day or by grouping of medication. In the preferred embodiment of the present invention, as shown in FIG. 18, the docking station 30 comprises a processing unit 31 wherein said processing unit 31 executes a machine learning software facilitated by an operating system 33, memory storage unit 32, a power supply 34, a data port adaptor 35 wherein said data port adaptor 35 corresponds to the data port adaptor 190 of the portable medicine container 100, a cellular system 36, a communication system 37, and a user interface 38.

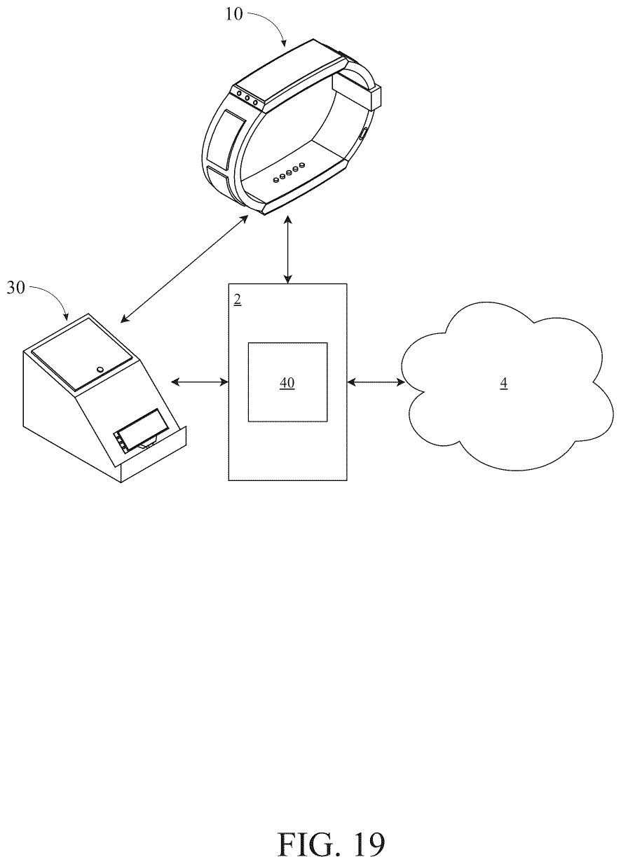

As shown in FIG. 19, in the preferred embodiments of the present invention machine learning and A.I. enabled medical device system 1 the portable medicine device 10 communicates to the mobile application 40 in the mobile device 2 and docking station 30 when the portable medicine container 100 is removably coupled to the docking station 30. In some embodiments of the present invention, the cloud 4 maintains the plurality of machine learning models supported by advanced A.I. tools with wider datasets wherein at least one said machine learning model is accessible on the cloud 4 through wireless communication between the cloud 4 and the mobile device 2 comprising the mobile application 40.

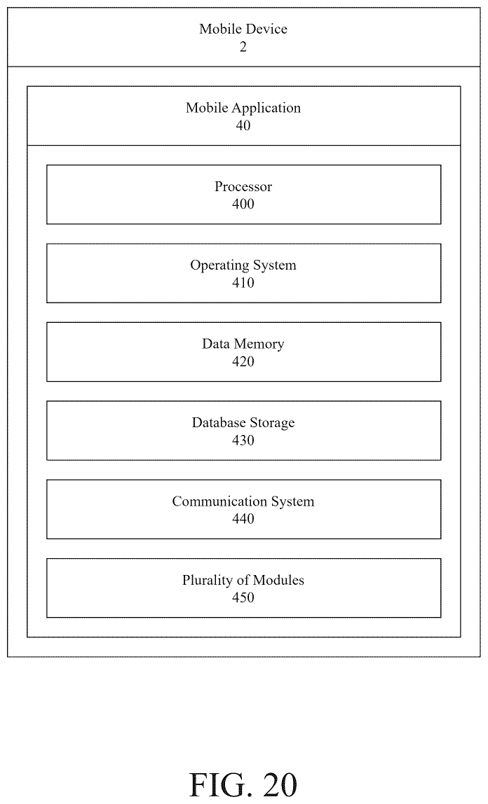

As shown in FIG. 20, in the preferred embodiment of the present invention, the mobile device 2 comprises of a mobile application 40 with user interfaces. In the preferred embodiment of the present invention, the mobile device 2 and mobile application 40 comprise a processor 400, an operating system 410, a data memory unit 420, a database storage 430, a communication system 440, and a plurality of modules 450. In the preferred embodiment of the present invention, the database storage 430 stores a collection of data gathered by the plurality of sensors 160 of the portable medicine device 10 of the machine learning and A.I. enabled medical device system 1 and user inputs in the plurality of modules 450 of the mobile application 40.

In the preferred embodiment of the present invention, data gathered from the at least one biometric sensor 161 and data gathered from the plurality of sensors inside the medicine compartment are collected from the machine learning and A.I. enabled medical device system 1 and user inputs from the mobile application 40 modules on real-time basis are sent to the machine learning device 200 of the portable medicine device 10 and model analyzer 1150. In the preferred embodiment of the present invention, the data collected by the plurality of sensors 160 in combination with the series of user inputs into the mobile application 40 are inputted into the holistic prescription machine learning model generator in the machine learning device 200, thereby training the holistic prescription machine learning model generator; in the preferred embodiment of the present invention, all the trained holistic prescription machine learning models for the user with biometric parameters are transmitted to the mobile application 40 will be further refined and trained by A.I. assistant 4601 in an analyzer module 460 of the mobile application 40 with feedback inputs; in the preferred embodiment of the present invention, all the retrained holistic prescription machine learning models from the Mobile application of the user with biometric parameters are transmitted back to the portable medicine device 10 storage unit. In the preferred embodiment of the present invention, the data collected by the plurality of sensors 160 in combination with the series of user inputs into the mobile application 40 are inputted into the medicine identifier machine learning model generator in the machine learning device 200, thereby training the medicine identifier machine learning model generator; in the preferred embodiment of the present invention, all the trained medicine identifier machine learning models with medicine identifier parameters are transmitted to the mobile application 40 will be further refined and trained by A.I. assistant 4601 in the analyzer module 460 of the mobile application 40 with feedback inputs; in the preferred embodiment of the present invention, all the retrained medicine identifier machine learning models with medicine identifier parameters are transmitted back to the portable medicine device 10 storage unit. In the preferred embodiment of the present invention, model analyzer 1150 executes series of computer executed holistic prescription selection and medicine identifier process method using processing unit 120 based on user's collected real-time biometric data and choose the most appropriate approved holistic prescription model dynamically from the plurality of trained and approved holistic prescription models that are deployed in the portable medicine device 10 storage unit as per the real-time healthcare needs of the device user. In the preferred embodiment of the present invention, model analyzer 1150 executes series of computer executed holistic prescription selection and medicine identifier process method using processing unit 120 based on collected real-time medicine identifier data and choose the most appropriate medicine identifier model dynamically from the plurality of trained and approved medicine identifier models that are deployed in the portable medicine device 10 storage unit. In the preferred embodiment of the present invention, model analyzer 1150 will pass on the selected holistic prescription control parameters to the device manger in the portable medicine device 10 which will reset the medicine administration schedule of the indicator interface 110, interactive display 170, and locking mechanism 1162 of the portable medicine device 10 as per the chosen holistic prescription model parameters and operates medicine administration functionality of the portable medicine device 10. Additionally, in the preferred embodiment of the present invention, transmitter 151, receiver 152 and the communication system are used to transmit and receive data of the portable medicine device 10 which facilitates the outputs indicative of the holistic prescription schedule and medicine administration of the machine learning and A.I. enabled medical device system 1.

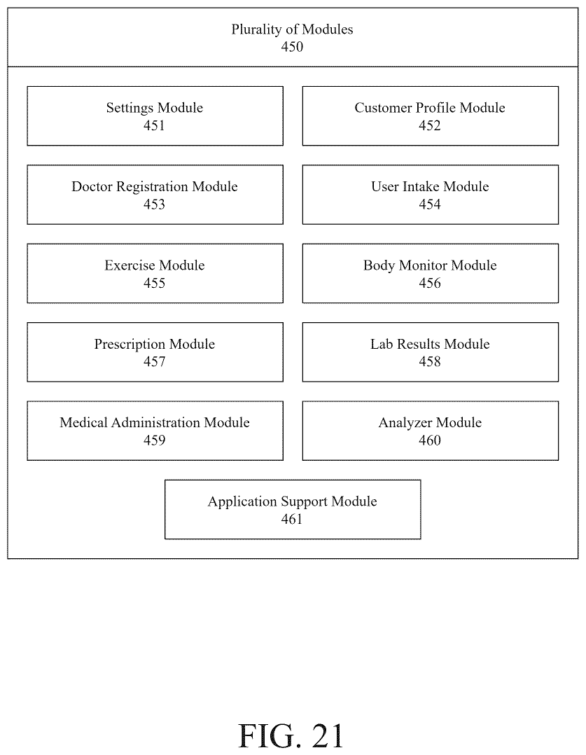

In the preferred embodiment of the present invention, the plurality of modules, as shows in FIG. 21, comprises a settings module 451, a customer profile module 452, a doctor registration module 453, a user intake module 454, an exercise module 455, a body monitor module 456, a prescription module 457, a lab results module 458, a medical administration module 459, an analyzer module 460, an application support module 461 and modules further facilitating the holistic prescription and medicine administration of the device. In the context of the present invention, each of the plurality of modules 450 facilitates user inputs and automatic parameter updates upon a series of inputs to the mobile application 40.

In the preferred embodiment of the present invention, the settings module 451 comprises of geolocation data, a calendar with date & time data, device communication setup data, portable medicine device 10 configuration data, machine learning and A.I. enabled medical device configuration data, and security data.

Furthermore, in the preferred embodiment of the present invention, the customer profile module 452, comprises user data including physical data such as BMI, height, weight, racial information, and other user health related parameters and contact information.

In the context of the present invention, the doctor registration module 453 comprises contact information for a medical professional. In the preferred embodiment of the present invention, the medical professional is a third-party user wherein the machine learning and artificial intelligence enabled medical device system 1 may receive input data from said medical professional based on their approvals of the trained holistic prescription machine learning models and manipulate the holistic prescription schedule & instructions. Examples of medical professionals include primary doctors, specialist doctors, and related medical professionals of the like. In some embodiments of the present invention, the doctor registration module 453 comprises data pertaining to a plurality of medical professionals.

In context of the present invention, the user intake module 454 is a collection of user consumption data. In the preferred embodiment of the present invention, consumption data comprises data pertaining to the reported events of food consumption data, supplements intake data and nutrition intake data. For example, in the context of the present invention, the user intake module 454 is populated with a user's food consumption for a meal, such as lunch, wherein the calorific value and nutrition data such as carbohydrates, sodium, potassium, vitamins and other nutritional fact data is recorded, the recommended values of the meal, a determination of compliance, and a data value pertaining to a metric indicating user health parameter responses and margin of error. In the context of the present invention, a margin of error is the difference between recommended and actual values. Furthermore, in the preferred embodiment, user intake module 454 is populated with data from the user or populated automatically from the sensor data.

In the context of the present invention, the user exercise module 455 is a collection of physical activity data performed by the user. In such embodiments, the user exercise module 455 is populated with date and time data, physical activity data including the type of exercises performed by the user, recommended exercises, actual exercises performed by the user, a determination of compliance, and a metric indicating user health parameter responses and a margin of error. Furthermore, in the preferred embodiment, the exercise module 455 is either populated with data from the user or populated automatically with sensor data.

In the preferred embodiment of the present invention, the body monitor module 456 is populated with data gathered by the plurality of sensors 160. Furthermore, in some embodiments of the present invention, a body monitor module 456 comprises a collection of data from a plurality of devices wherein said plurality of devices comprises at least one portable medicine device 10 and a third-party device.

In the preferred embodiment of the present invention, the prescription module 457 is a collection of data values, wherein said data values are collected by the plurality of sensors 160 including the camera 1174, the dosage sensor 1172, and the lidar scanner 1173. Furthermore, in the preferred embodiment of the present invention, a series of user inputs into the prescription module 457 provides a computer executable process by which said process facilitates an output generated by the indicator interface 110 in accordance with the predetermine schedule. Furthermore, in the preferred embodiment of the present invention, the mobile application 40, specifically the prescription module 457, and the respective inputs populated by the plurality of sensors 160 and user inputs, communicates to a user through the interactive display with an interactive display 170 screen which receives inputs pertaining to the holistic prescription model, information pertaining to a medication contained within the medicine container 100 and intake instructions, wherein such embodiments, the locking mechanism 1162 adjustably engages dependent on the holistic prescription schedule, thereby permitting access to the interior cavity 1170 of the medicine container 100. In the preferred embodiment of the present invention, an identification of the medication within the interior cavity 1170 is populated within the prescription module 457.

Moreover, in regard to the prescription module 457, in the preferred embodiment of the present invention, said module comprises a collection of data wherein said data pertains to the medication contained within the interior cavity 1170 of a first portable medicine container 100 with the holistic prescription schedule pertaining to the medication contained within the first portable medicine container 100, the medication contained within the interior cavity 1170 of a second portable medicine container 100 with the holistic prescription schedule pertaining to the medication contained within the second portable medicine container 100.

In the context of the present invention, the holistic prescription schedule is a medication administration schedule, intended to be given from a medical professional to the user, wherein said medication administration schedule is a dosage regimen meant for the systematize dosage consumption of a medication per unit of time. In the preferred embodiment of the present invention, the portable medicine device 10, notifies the user of the holistic prescription schedule through outputs generated by the interactive display and indicator interface 110, indicative of actionable tasks intended to be performed by the user. For example, a medical professional prescribes a holistic prescription medication instructions wherein the prescribed medication is contained within the interior cavity 1170 of the portable medicine device 10, whereby the timer 112 regulates a time in accordance to the holistic prescription schedule, the alarm 111 generates an output indicative of the intended time in which the medication is to be administered, the locking mechanism 1162 disengages allowing access to the medication within the interior cavity 1170, while the at least one light 1171 generates a series of outputs representative of ongoing processes. In the context of the present invention, a colored illumination output generated by at least one light 1171 may, in some embodiments, indicate a time to administer the medication, a time between dosages, and a time to refill the medicine container 100. In the preferred embodiments of the present invention, a green light is indicative of medicine administration, a yellow light is indicative of a stand-by period wherein the indicator interface 110 is indicative of a waiting period between dosages, and a red light is indicative of an empty medicine container 100, thereby notifying the user that medication is not present within the interior cavity 1170 and indicates time for refill.

In the preferred embodiment of the present invention, the lab results module 458 comprises a collection of data pertaining to medical examination and related lab test results of the user. In the preferred embodiment of the present invention, the collection of data contained within the lab results module 458 is populated through at least one user input or by auto sensor input.

In context of the present invention, the medicine administration module 459 is a collection of user's medicine administration data. In the preferred embodiment of the present invention, consumption data comprises data pertaining to the prescribed medication, a date & time data value, a dosage data value, a compliance data value and a margin of error value. Furthermore, in the context of the present invention, the medicine administration module 459 is a historical record of the user's medicine administration and dosage data of the prescribed medication.

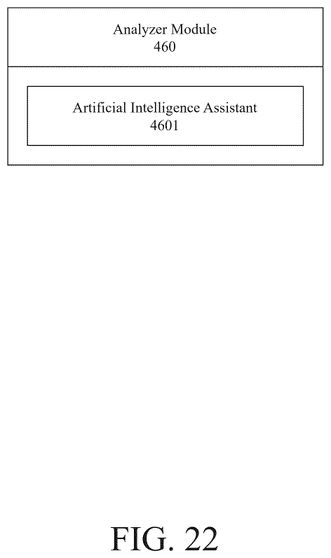

In the context of the present invention, as shown in FIG. 22, the analyzer module 460 comprises an artificial intelligence assistant 4601 comprising of holistic prescription machine learning model analyzer 1150 tool, a medicine identifier machine learning model analyzer 1150 tool is executed in the mobile processing system to retrain and refine holistic prescription machine learning models and medicine identifier machine learning models sent by the Model analyzer 1150 of the portable medicine device. Furthermore, in the preferred embodiment of the present invention, user input and auto populated data from the customer profile module 452, user intake module 454, exercise module 455, body monitor module 456, prescription module 457, lab results module 458 and medical administration module 459, along with plurality of sensor data, user response pattern, is utilized by the machine learning device 200 in the portable medicine device 10 to train and generate the holistic prescription machine learning model, thereby generating user specific holistic prescription model recommendations for the user. Furthermore, in the preferred embodiment of the present invention the generated holistic prescription and medicine identifier machine learning models by the machine learning device 200 in the portable medicine device 10 is transmitted to the mobile application 40 for further refined by the A.I. assistant 4601 in the analyzer module 460 of the mobile application 40. Furthermore, in the preferred embodiment of the present invention, user input into the analyzer module 460 determines the privileges of the machine learning model analyzer 1150 tool wherein user input into the analyzer module 460 control the information from other plurality of modules that is utilized in training the holistic prescription machine learning models and medicine identifier machine learning models. The user input into the analyzer module 460 further determines the processes and timelines of the machine learning and A.I. enabled medical device system 1 as they pertain to collection, processing, and communication of data through user input and data gathered by the plurality of sensors 160. In the context of the present invention, the trained holistic prescription models of the user with biometric parameters are transmitted after approval, from the mobile application 40 and deployed to the portable medicine device 10 data storage unit. In the context of the present invention, all the trained holistic prescription models with biometric parameters are transmitted from the analyzer module 460 to the cloud 4 database storage for further model generation with wider data sets. In the context of the present invention, all the cloud 4 trained holistic prescription models with biometric parameters are transmitted to the analyzer module 460 of the mobile application 40 executed by the mobile processing unit 400 facilitated by the operating system 410, data memory 420, communication system 440, plurality of modules 450 of the mobile device 2 for further refinement of the holistic prescription machine learning model generator. In the context of the present invention, the machine learning and A.I. enabled medical device utilizes all the mobile application 40 module input data along with real-time user health biometric data to create plurality of holistic prescription models and gets refined by the A.I. assistant 4601 in the mobile application 40 before deploying the approved machine learning models to the portable medicine device 10 storage unit. In some embodiments of this invention data pertaining to the mobile application 40 modules can be collected by the portable medicine device 10 with its graphical user interface through the interactive display.

Regarding the app support module 461, within the context of the present invention, the application support module 461 facilitates and comprises a collection of software programs which controls the core data collections, processing, authorizations, the device computer method updates, the mobile application 40 updates, reporting & upgrade tools. Furthermore, in the preferred embodiment of the present invention, the app support module 461 further provides a version control and processes which facilitate the machine learning and A.I. enabled medical device system 1 integration with the mobile device 2, mobile application 40, the docking station 30, cloud 4 and the computer executable method 3.

Referring to FIG. 8, in the preferred embodiment of the present invention, the computer executed method 3 comprises a holistic prescription machine learning model generator method 50, a medicine identifier machine learning model generator method 60, and a holistic prescription selection and medicine identifier process method 70. In the context of the present invention, the holistic prescription machine learning model generator method 50 is a computer executable processes comprising a series of methods, executed by a machine learning software 201 in the machine learning device 200 and facilitated by the operating system 140 and memory storage unit 130,202, wherein said machine learning software executes the holistic prescription machine learning model generator method 50 in the processing unit 120 using the at least one biometric sensor 160 data and user input data of mobile application 40, wherein data is utilized to train the holistic prescription machine learning generator to develop holistic prescription models wherein the trained and approved holistic prescription models along with the model parameters are deployed in the portable medicine device 10 storage unit. Furthermore, within the context of the present invention, the medicine identifier machine learning model generator method 60 is a computer executable process comprising of a series of methods, executed by a machine learning software 201 in the machine learning device 200 and facilitated by the operating system 140 and memory storage unit 130,202, wherein said machine learning software executes medicine identifier machine learning model generator method 60 in the processing unit 120 using the at least one medicine identifier sensor 160 data and user input data of mobile application 40, wherein data is utilized to train the medicine identifier machine learning generator method 60 to develop medicine identifier models using collected sensor data and deploy the trained and approved medicine identifier models in the portable medicine device 10 storage unit. Furthermore, within the context of the present invention, the holistic prescription selection and medicine identifier process method 70 is a computer executable process comprising a series of methods, executed and facilitated by the model analyzer 1150 in the portable medicine device 10. The model analyzer 1150 executes holistic prescription selection and medicine identifier process method 70 is a computer executable process comprising of a series of methods, executed by the model analyzer software 1151 in the model analyzer 1150 by the processing unit 120 and facilitated by the operating system 140 and memory storage unit 130,1152, and control the plurality of sensors 160 to collect real-time device sensor data to accurately identify the medicine inside the medicine container 100 of the portable medicine device 10 from the plurality of trained and approved medicine identifier models stored in the portable medicine device 10 storage unit. Furthermore, within the context of the present invention, model analyzer 1150 executes holistic prescription selection and medicine identifier process method which is included in the model analyzer 1150 software which is executed by the processing unit 120 and facilitated by the operating system 140 and memory storage unit 130,1152, and control the plurality of sensors 160 to collect the real-time biometric data and mobile application 40 user input data to dynamically determine the most appropriate holistic prescription model from the plurality of deployed holistic prescription machine learning models stored in the portable medicine device 10 storage unit. Furthermore, within the context of the present invention, model analyzer 1150 executes holistic prescription selection and medicine identifier process method to record user response pattern which is transmitted to holistic prescription machine learning model generator for further learning and training of holistic prescription machine learning model generator in the machine learning device 200. Furthermore, within the context of the present invention, model analyzer 1150 executes holistic prescription selection and medicine identifier process method and send the model parameters of the chosen holistic prescription model to the device manager 1140 to control the portable medicine device 10 interactive display 170, indicator interface 110 and device locking mechanism 1162 for the user to follow holistic prescription based on the selected holistic prescription model. Furthermore, within the context of the present invention, machine learning device 200 and model analyzer 1150 in the portable medicine device 10 controls plurality of relevant sensors in the device to collect real-time sensor data from the device and activate and deactivate the devices as needed by passing the control parameters to the device manager 1140. In the preferred embodiment of the invention, identifying the medicine availability in the medicine container 100 and holistic prescription instructions are synced up with both the mobile app and the machine learning and A.I. enabled medical device, ensuring that users have the correct medication information and the guidance need to take it correctly.

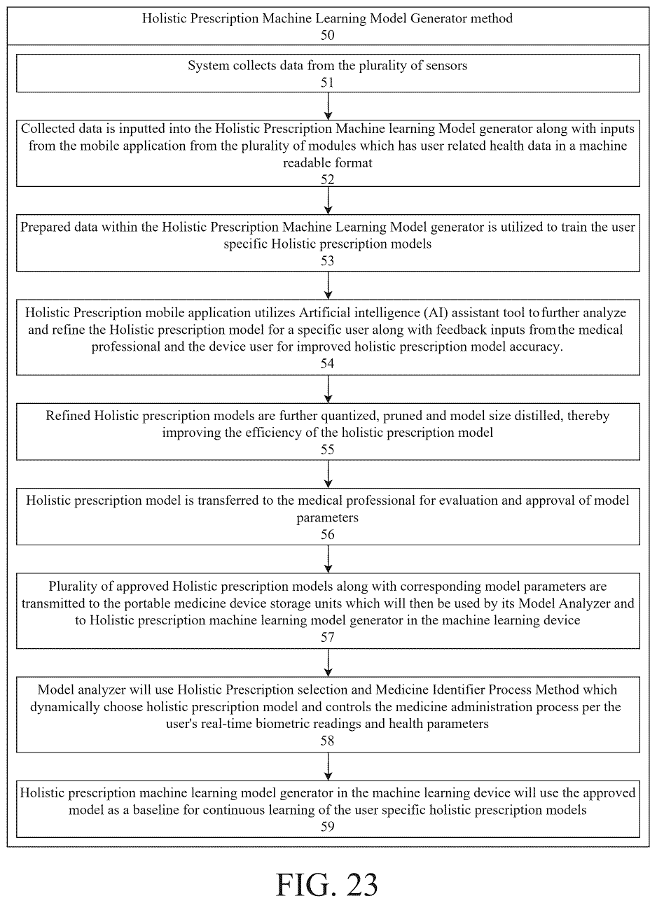

Referring to FIG. 23, in the preferred embodiment of the present invention, the holistic prescription machine learning model generator method 50 comprises a first step 51 wherein the plurality of sensors 160 of the machine learning and artificial intelligence enabled device system and portable medicine device 10 collects data. In a second step 52, the collected data is inputted into the holistic prescription machine learning model generator along with inputs from the machine learning and artificial intelligence enabled device system integrated holistic prescription mobile application 40 from its plurality of modules which has user related health data in a machine-readable format. In a third step 53, the prepared data within the holistic prescription machine learning model generator is utilized to train a user specific holistic prescription model. In a fourth step 54, holistic prescription mobile application 40 utilizes artificial intelligence (A.I.) Assistant to further analyze and refine the holistic prescription model for a specific user along with feedback inputs from the medical professional and the device user for improved holistic prescription model accuracy. In a fifth step 55, the refined holistic prescription models are further quantized, pruned and model size distilled, thereby improving efficiency of the holistic prescription model. In a sixth step 56, the holistic prescription model is transferred to the medical professional for evaluation and approval of model parameters. In a seventh step 57, a plurality of approved holistic prescription models along with corresponding model parameters are transmitted to the portable medicine device 10 storage units which will then be used by its model analyzer 1150 and to holistic prescription machine learning model generator in the machine learning device 200. In an eighth step 58, machine learning and artificial intelligence enabled portable medicine device 10 model analyzer 1150 will use holistic prescription selection and medicine identifier process method which dynamically choose holistic prescription model and controls the medicine administration process of the machine learning and A.I. enabled medical device as per the user's real-time biometric readings and health parameters. In a ninth step 59, machine learning and artificial intelligence enabled medicine device holistic prescription machine learning model generator in the machine learning device 200 will use the approved model as a baseline for continuous learning of the user specific holistic prescription models.

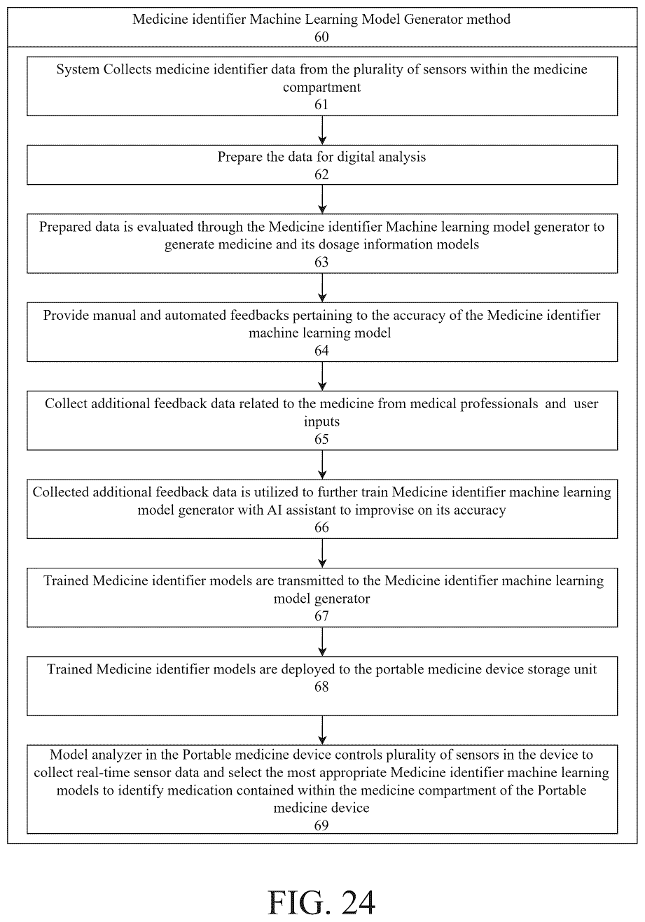

Referring to FIG. 24, in the preferred embodiment of the present invention, the medicine identifier machine learning model generator method 60 comprises a first step 61 wherein the plurality of sensors 160 within the medicine container 100 of the portable medicine device 10 collects medicine identifier data. In a second step 62, the collected data is prepared for digital analysis. In a third step 63, the prepared data is evaluated through the medicine identifier machine learning model generator to generate medicine and its dosage information models. In a fourth step 64, manual and automated feedback is provided as an input, wherein said feedback is indicative of the accuracy of the generated medicine identifier machine learning model. In a fifth step 65, collect additional feedback data related to the medicine from medical professionals and user inputs. In a sixth step 66, collected additional feedback data is utilized to further train medicine identifier machine learning model generator with A.I. assistant 4601 to improvise on its accuracy. In a seventh step 67, trained medicine identifier models are transmitted to the medicine identifier machine learning model generator. In an eighth step 68, trained medicine identifier models are deployed to the portable medicine device 10 storage unit. In a ninth step 69, model analyzer 1150 in the portable medicine device 10 controls plurality of sensors 160 in the device to collect real-time sensor data and select the most appropriate medicine identifier machine learning model to identify medication contained within the medicine container 100 of the portable medicine device 10.

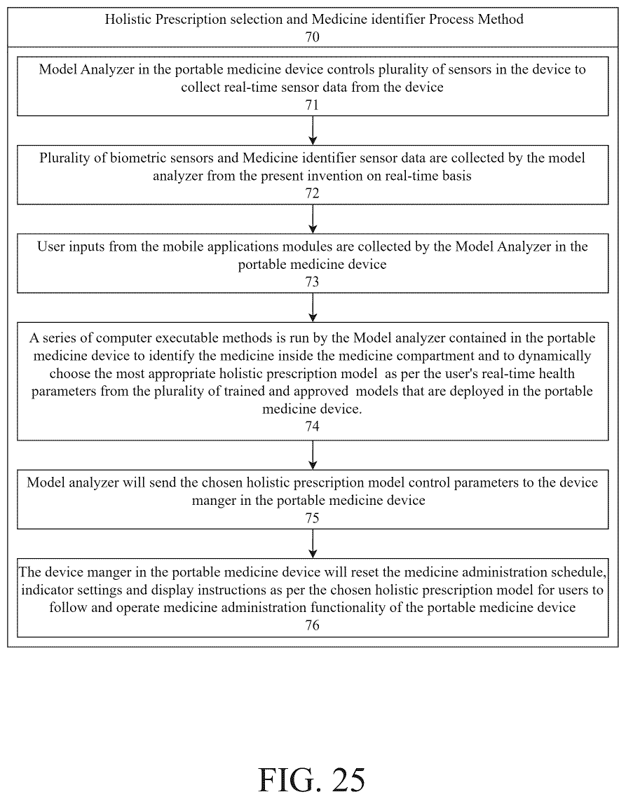

Referring to FIG. 25, in the preferred embodiment of the present invention, holistic prescription selection and medicine identifier process method 70 is executed by the model analyzer 1150 of the portable medicine device 10 which comprises a first step 71, model analyzer 1150 in the portable medicine device 10 controls plurality of sensors 160 in the device to collect real-time sensor data from the device. In a second step 72, plurality of biometric sensor 161s and medicine identifier sensor data are collected by the model analyzer 1150 from the machine learning and A.I. enabled medical device system 1 on real-time basis. In the third step 73, user inputs from the mobile application 40s modules are collected by the model analyzer 1150 in the portable medicine device 10. In the fourth step 74, a series of computer executable methods is run by the model analyzer 1150 contained in the portable medicine device 10 to identify the medicine inside the medicine container 100 and to dynamically choose the most appropriate holistic prescription model as per the user's real-time health parameters from the plurality of trained and approved models that are deployed in the portable medicine device 10. In the fifth step 75, model analyzer 1150 will send the chosen holistic prescription model control parameters to the device manager 1140 in the portable medicine device 10. In the sixth step 76, the device manger in the portable medicine device 10 will reset the medicine administration schedule, indicator settings and display instructions as per the chosen holistic prescription model for users to follow and operate medicine administration functionality of the portable medicine device 10.

Although the invention has been explained in relation to its preferred embodiment, it is to be understood that many other possible modifications and variations can be made without departing from the spirit and scope of the invention.

1. A machine learning and artificial intelligence enabled medical device system comprising:
a portable medicine device; and
a mobile application;
wherein:
the portable medicine device comprises:
an interior cavity;
an indicator interface;
a processing unit;
a plurality of sensors;
a machine learning device;
a device manager; and
a model analyzer;
the interior cavity configured to contain medication wherein said internal cavity comprises a door and a locking mechanism such that:
the door hingedly connects to the portable medicine device, providing adjustable access to the interior cavity; and
the locking mechanism selectively engaging and disengaging in accordance with a plurality of control parameters of a selected holistic prescription model such that engagement and disengagement of the locking mechanism selectively controls access to the interior cavity by permitting movement of the door;
the indicator interface generating an output indicative of information pertaining to the interior cavity;
the processing unit receiving computer readable data collected from the plurality of sensors, executing a series of computer executable methods, and communicating signals throughout the machine learning and artificial intelligence enabled medical device system;
the plurality of sensors comprising at least one biometric sensor and at least one environmental sensor;
the machine learning device comprising a machine learning software comprising a series of holistic prescription model parameters in which data collected by the plurality of sensors and an at least one user input from the mobile application is processed to generate an at least one machine learning model;
the model analyzer utilizing data collected by the plurality of sensors and user inputs from the mobile application to analyze and select the at least one machine learning model;
the model analyzer sends a plurality of control parameters of a selected holistic prescription model to a device manager;
the device manager actuates the locking mechanism and the indicator interface in accordance with the plurality of control parameters;
the mobile application is a computer software composed of a plurality of computer executable methods wherein said mobile application communicates with the portable medicine device and facilitates the at least one machine learning model; and
the mobile application comprises an artificial intelligence (A.I.) assistant.

2. The machine learning and artificial intelligence enabled medical device system as claimed in claim 1, wherein the interior cavity comprises:
a light;
a dosage sensor;
a LiDAR scanner;
a camera; and
a climate controller;
wherein:
the light illuminates the interior cavity;
the camera receives visual data from within the interior cavity;
the dosage sensor coordinates with the machine learning device and model analyzer to obtain data from within the interior cavity, wherein such data pertains to the medication dosage; and
the LiDAR scanner coordinates with the camera to detect medication within the interior cavity.

3. The machine learning and artificial intelligence enabled medical device system, as claimed in claim 2, wherein the at least one biometric sensor comprises at least one of:
a heart rate/pulse sensor;
a blood oxygen sensor;
a body temperature sensor;
a respiration sensor;
a bodily activity sensor;
a sleep pattern sensor;
a sweat sensor;
a stress sensor;
a hydration sensor;
a BMI sensor;
a UV radiation sensor;
a blood sugar sensor;
a urea level sensor;
a creatine level sensor;
a lactate level sensor;
a calorific sensor;
a body vitals sensor; and
a biometric sensor of the like.

4. The machine learning and artificial intelligence enabled medical device system, as claimed in claim 3, wherein the at least one environmental sensor comprises at least one of:
a weather sensor;
a temperature sensor;
a humidity sensor;
a motion sensor;
an image scanner;
a LiDAR sensor; and
an environmental sensor of the like.

5. The machine learning and artificial intelligence enabled medical device system, as claimed in claim 1, wherein the indicator interface comprises:
an alarm;
a timer;
an at least one indicator light; and
a speaker;
wherein:
the timer controls a series of outputs generated by the alarm and the at least one indicator light;
the alarm generates an output indicative of an actionable task intended to be performed by the user in accordance with the plurality of control parameters; and
the at least one indicator light generates an output indicative of a status wherein said status is representative of an availability of medicine contained within the interior cavity.

6. The machine learning and artificial intelligence enabled medical device system, as claimed in claim 2, wherein the door comprises an interactive display whereby said interactive display receives an input pertaining to the at least one holistic prescription model parameter.

7. The machine learning and artificial intelligence enabled medical device system, as claimed in claim 2, wherein the processing unit facilitates storage and processing of data obtained by the plurality of sensors to a memory storage unit.

8. The machine learning and artificial intelligence enabled medical device system, as claimed in claim 7, wherein the portable medicine device further comprises a communication device, a location proximation system, and a cellular system.

9. The machine learning and artificial intelligence enabled medical device system, as claimed in claim 1, wherein the portable medicine device is composed of a medicine container and a wearable mount wherein the medicine container is removably attachable to the wearable mount.

10. The machine learning and artificial intelligence enabled medical device system, as claimed in claim 4, wherein the mobile application comprises a plurality of modules including:
a settings module;
a customer profile module;
a doctor registration module;
a user intake module;
an exercise module;
a body monitor module;
a prescription module;
a lab result module;
a medical administration module;
an analyzer module; and
an application support module
wherein:
each of the plurality of modules facilitates user modification and automatic modification through transmission of data collected by the plurality of sensors upon a series of inputs to the mobile application.

11. The machine learning and artificial intelligence enabled medical device system, as claimed in claim 4, wherein:
the plurality of sensors collect data;
the machine learning device processes data collected by the plurality of sensors and data received from the mobile application;
the at least one holistic prescription model is trained using data processed by the machine learning device and transmitted to the mobile application;
the A.I. assistant analyzes the holistic prescription model in conjunction with an input received in the mobile application, refining the holistic prescription model;
the portable medicine device receives the refined holistic prescription model; and
the model analyzer and machine learning device execute the refined prescription model through the portable medicine device.

12. The machine learning and artificial intelligence enabled medical device system, as claimed in claim 10, wherein:
the plurality of sensors collect data;
the machine learning device evaluates the data collected by the plurality of sensors;
a medicine identifier model is trained using data collected by the plurality of sensors through the machine learning device;
the medicine identifier model is communicated to the mobile application;
the A.I. assistant analyzes the medicine identifier model in conjunction with an input received in the mobile application, thus refining the medicine identifier model;
the refined medicine identifier model is communicated to the portable medicine device;
the model analyzer controls the plurality of sensors within the portable medicine device; and
the model analyzer determines an identification of medicine within the interior cavity of the portable medicine device.

13. The machine learning and artificial intelligence enabled medical device system, as claimed in claim 1, wherein:
the model analyzer controls the plurality of sensors to collect real-time data;
the at least one biometric sensor communicates data to the model analyzer;
the mobile application receives a plurality of inputs through the plurality of modules and communicates said input to the model analyzer;
the model analyzer sends the plurality of control parameters to the device manager; and
the device manager actuates the locking mechanism and the indicator interface, and controls operation of an interactive display, in accordance with the plurality of control parameters.

14. (canceled)

15. (canceled)

16. (canceled)

17. (canceled)

18. (canceled)

19. (canceled)

20. (canceled)

21. The machine learning and artificial intelligence enabled medical device system, as claimed in claim 1 wherein the at least one holistic prescription model parameter comprises at least one of: an instruction on the type of dietary and nutritional values of the food intake, an instruction on the supplemental medications and its intake methods, an instruction on the combination of medication intake, an instruction on the variable medicine dosages and medicine intake details, an instruction on the variable timings of the medicine intake, an instruction on the type and the amount of exercise to be done, an instruction on the stress reduction and sleep pattern improvements, an instruction on adjusting environmental factors, an instruction on the alternate treatment plans, and combinations thereof.

22. The machine learning and artificial intelligence enabled medical device system, as claimed within claim 1, wherein the device manager controls access to the interior cavity by actuating the locking mechanism in accordance with the plurality of control parameters provided by the model analyzer, the plurality of control parameters being derived from real-time biometric sensor inputs in combination with user inputs received by the mobile application and determined in accordance with a selected holistic prescription model.

23. The machine learning and artificial intelligence enabled medical device system, as claimed in claim 1, wherein the device manager controls access to the interior cavity by engaging the locking mechanism when a holistic prescription schedule designates a locked state and by disengaging the locking mechanism when the holistic prescription schedule designates an unlocked state.
